SEQUENCE LISTING (8 sequences)

SEQ ID NO: 1 - DNA, 16 bp, Artificial Sequence
  Description of Artificial Sequenceexemplary retinoic acid-related orphan receptor alpha (Rora) response element
gtaggtcatgacctac

SEQ ID NO: 2 - DNA, 10 bp, Artificial Sequence
  Description of Artificial Sequenceretinoic acid-related orphan receptor (ROR) element (RORE) sequence
awntrggtca

SEQ ID NO: 3 - DNA, 33 bp, Artificial Sequence
  Description of Artificial SequenceExpand Long Template PCR system amplification primer for Bmal1 promoter
gatcgagctcgggacgacggcgagctcgcagag

SEQ ID NO: 4 - DNA, 31 bp, Artificial Sequence
  Description of Artificial SequenceExpand Long Template PCR system amplification primer for Bmal1 promoter
gatcctcgagcgcacccgcactcggatcccg

SEQ ID NO: 5 - DNA, 28 bp, Artificial Sequence
  Description of Artificial SequenceBmal1 promoter RORE element wild-type complementary probe oligo
gaaggcagaaagtaggtcagggacggag

SEQ ID NO: 6 - DNA, 28 bp, Artificial Sequence
  Description of Artificial SequenceBmal1 promoter RORE element wild-type complementary probe oligo
ctccgtccctgacctactttctgccttc

SEQ ID NO: 7 - DNA, 28 bp, Artificial Sequence
  Description of Artificial SequenceBmal1 promoter RORE element mutant complementary probe oligo
gaaggcagaaagtacgtcagggacggag

SEQ ID NO: 8 - DNA, 28 bp, Artificial Sequence
  Description of Artificial SequenceBmal1 promoter RORE element mutant complementary probe oligo
ctccgtccctgacgtactttctgccttc

(19) United States
(12) Patent Application Publication
(10) Pub. No.: US 2005/0186138 A1
(43) Pub. Date: Aug. 25, 2005

(54) Methods and compositions for modulating circadian rhythm

(75) Inventors: John B. Hogenesch, Encinitas, CA (US); Trey Kyle Sato, La Jolla, CA (US); Steve A. Kay, San Diego, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: IRM, LLC, Hamilton (BM); The Scripps Research Institute, La Jolla, CA (US)

(21) Appl. No.: 10/965,607

(22) Filed: Oct. 13, 2004

Related U.S. Application Data
(60) Provisional application No. 60/512,422, filed on Oct. 16, 2003.

(51) Int. Cl. A61K 49/00

(52) U.S. Cl. 424/9.2

(57) ABSTRACT

The present invention provides methods of identifying circadian rhythm modulators and methods of modulating circadian rhythm in animals.

### CROSS-REFERENCE TO RELATED PATENT APPLICATIONS

The present application claims benefit of priority to U.S. Provisional Patent Application No. 60/512,422, filed Oct. 16, 2003, which is incorporated in its entirety by reference for all purpose.

### STATEMENT AS TO RIGHTS TO INVENTIONS MADE UNDER FEDERALLY SPONSORED RESEARCH AND DEVELOPMENT

This invention was made with Government support under Grant No. MH51573 and DK064086 awarded by the National Institutes of Health. The government has certain rights in this invention.

### BACKGROUND OF THE INVENTION

The circadian clock plays an integral role in timing rhythmic behavior, such as the consolidation of locomotor activity and physiology with anticipated daily environmental changes. In mammals, the core oscillator resides within the hypothalamic suprachiasmatic nucleus (SCN), which can maintain circadian rhythms in the absence of synchronizing (or entraining) light input (Reppert, S. M. & Weaver, D. R. Nature 418:935-41 (2002)). The transcriptional activators, Clock and Bmal1/Mop3, heterodimerize on E-box DNA elements (CACGTG) within the promoters of the repressor Period and Crytpochrome genes. See, e.g., Gekakis, N. et al. Science 280:1564-9 (1998); Hogenesch, J. B., et al. Proc Natl Acad Sci USA 95:5474-9 (1998); Etchegaray, J. P., et al. Nature 421:177-82 (2003). As the Per and Cry mRNA and proteins levels rise, they bind Clock/Bmal1 to repress their own transcription, thereby forming a 24 hr long negative feed-back loop, the general mechanism that is conserved throughout clock-abiding organisms. See.e.g., Panda, S., Hogenesch, J. B. & Kay, S. A. Nature 417:329-35 (2002); Young, M. W. & Kay, S. A. Nat Rev Genet 2:702-15 (2001); Dunlap, J. C. Cell 96, 271-90 (1999).

Disruption of circadian rhythms can result in a number of pathophysiological states in humans. The most common of these pathophysiological states is jet lag, though a number of other sleep or circadian rhythm disorders also occur. Therefore, there is a need in the art for methods of modulating circadian rhythms in mammals to treat these conditions. The present invention addresses this and other problems.

### BRIEF SUMMARY OF THE INVENTION

The present invention provides methods for identifying a therapeutic agent for modulating circadian rhythm in an animal. In some embodiments, the method comprises (i) identifying an agent that modulates Rora activity or expression; (ii) testing the identified agent for an effect on the regulation of circadian rhythm in the animal; and (iii) selecting an agent that modulates the regulation of circadian rhythm in the animal.

In some embodiments, Rora is selected from the group consisting of human Rora1, human Rora2, human Rora3, and human Rora4. In some embodiments, the agent increases Rora expression. In some embodiments, the agent increases Rora activity. In some embodiments, the agent decreases Rora expression. In some embodiments, the agent decreases Rora activity.

In some embodiments, Rora activity is measured by determining the expression from Bmal1 promoter. In some embodiments, the Bmal1 promoter is operably linked to a reporter polynucleotide. In some embodiments, the animal is a mouse.

The present invention also provides methods of modulating circadian rhythm in a mammal in need thereof. In some embodiments, the method comprises administering to the mammal an effective amount of a Rora modulator.

In some embodiments, the modulator is by a method comprising the steps of identifying an agent that modulates Rora activity or expression; testing the identified agent for an effect on the regulation of circadian rhythm in the animal; and selecting an agent that modulates the regulation of circadian rhythm in the animal, thereby identifying a modulator of circadian rhythm.

In some embodiments, timing of administration of the selected agent is pre-determined to coincide with an appropriate phase of an existing circadian rhythm to produce a selected modulation of the circadian rhythm. In some embodiments, the selected agent is used to treat or prevent a sleep disorder. In some embodiments, the mammal has a condition selected from the group selected from insomnia, Seasonal Affective Disorder, Shift Work dysrhythmia, delayed-sleep phase syndrome, and jet-lag. In some embodiments, the mammal is a human.

In some embodiments, the selected agent is administered in conjunction with melatonin or a compound that suppresses or stimulates melatonin production. In some embodiments, the selected agent is administered in conjunction with light therapy.

### Definitions

The term “Rora” refers to the polypeptide or polynucleotide encoding the polypeptide retinoic acid-related orphan receptor alpha (also known as ROR alpha). Human Rora isoforms include, Rora1, Rora2, Rora3 and Rora4. See, e.g., Becker-Andre et al. Biophys. Res. Commun. 194:1371-1379 (1993); Giguere, et al., Genes Dev. 8:538-553 (1994); Hamilton et al., Nature 379:736-739 (1996); Matysiak-Scholze, et al., Genomics 43:78-84 (1997). Exemplary Rora polypeptides include those displayed in Genbank accession numbers NP_{—}002934.1, NP_{—}599022.1, NP_{—}599023.1 and NP_{—}599024.1 and variants, SNPs and fragments thereof. Other Rora polypeptides include, e.g., the bovine protein BT3446 (Genbank accession number AV613403.1) and the mouse protein depicted in AK034375 as well as variants, SNPs and fragments thereof.

“Rora modulators” are used herein to refer to inhibitory or activating molecules of melanopsin expression or activity. Inhibitors are agents that, e.g., inhibit expression of Rora or bind to, partially or totally block stimulation, decrease, prevent, delay activation, inactivate, desensitize, or down regulate the activity of Rora, e.g., antagonists. Activators are agents that, e.g., induce or activate the expression of Rora or bind to, stimulate, increase, open, activate, facilitate, or enhance activation, sensitize or up regulate the activity of Rora, e.g., agonists. Modulators include naturally occurring and synthetic ligands, antagonists, agonists, small chemical molecules and the like. Assays for inhibitors and activators include, e.g., applying putative modulator compounds to cells expressing a polypeptide of the invention and then determining the functional effects on Rora activity. Samples or assays comprising Rora that are treated with a potential modulator are compared to control samples without the modulator to examine the extent of effect. Control samples (not treated with modulators) are assigned a relative activity value of 100%. Inhibition of Rora is achieved when the Rora activity value relative to the control is less than about 80%, optionally 50% or 25, 10%, 5% or 1%. Activation of Rora is achieved when Rora activity value relative to the control is at least 110%, optionally 150%, optionally 200, 300%, 400%, 500%, or 1000-3000% or more higher.

A “circadian rhythm” refers to an internal daily biological clock in an organism. Typically circadian rhythms oscillate with an approximate 24 hour periodicity.

A “circadian rhythm phase shift” refers to a change in the phase of locomotor of an animal, typically in response to a perturbation in the animal's internal clock. When a perturbation is applied to an animal, it is common to observe that the time at which an event occurs (i.e. the phase) is often different than a control that did not receive the perturbation. This phase shift is usually measured in hours or minutes from the control (or in degrees from a 360° cycle or in circadian time). The magnitude of the phase shift usually depends on the time in the cycle at which the perturbation was applied. The phase shift of two animals can be compared by providing the same perturbation to the light/dark cycle of the animals and then measuring a change in phase shift. Phase shift of the first animal (e.g., a melanopsin knockout animal) is attenuated compared with the phase shift of second animal (e.g., a wild type or other control animal) if the phase shift observed for the first animal is less than the phase shift observed for the second animal. Phase shift can be measured as a percentage of the control. Exemplary attenuated phase shifts can be at least 5%, 10%, 15%, 20%, 25%, 30%, 35%, 40%, 45%, 50%, 60%, 70%, 80%, 90% or 95% of the control.

“Light therapy” refers to exposure of a subject to light with the goal of supplementing the amount of light a subject normally receives. Light therapy is can be used to treat such disorders and SADS, which is caused by long, dark winters.

The term “nucleic acid” or “polynucleotide” refers to deoxyribonucleotides or ribonucleotides and polymers thereof in either single- or double-stranded form. The term nucleic acid is used interchangeably with gene, cDNA, and mRNA encoded by a gene.

The terms “polypeptide,” “peptide” and “protein” are used interchangeably to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M) (see, e.g., Creighton, Proteins (1984)).

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (e.g., a polypeptide of the invention), which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same sequences. Two sequences are “substantially identical” if two sequences have a specified percentage of amino acid residues or nucleotides that are the same (i.e., 60% identity, optionally 65%, 70%, 75%, 80%, 85%, 90%, or 95% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides in length, or more preferably over a region that is 100 to 500 or 1000 or more nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Two examples of algorithms that are suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores -are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

### DETAILED DESCRIPTION OF THE INVENTION

I. Introduction

The present invention is based, in part, on the surprising discovery that Rora plays a role in regulating circadian rhythm. This discovery allows for the identification of novel molecules useful for altering circadian rhythm in a subject. In addition, Rora modulators can be used to modulate circadian rhythm in subjects.

II. Circadian Rhythm Modulators

Modulators of Rora are useful for preventing or treating a number of conditions by specifically advancing or delaying the phase of circadian rhythms in humans. The administration to a subject of an appropriate amount of a modulator of the invention is useful, for example, to achieve chronobiologic effects and/or to alleviate circadian rhythm phase disturbances in subjects in need thereof. Conditions treatable by such modulators include, e.g., insomnia, Seasonal Affective Disorder (SAD), Shift Work dysrhythmia, delayed-sleep phase syndrome (in which the major sleep episode is delayed by 2 or more hours of the desired bedtime), Irregular Sleep/Wake Pattern (characterized by irregular sleep/wake timing in which napping is prevalent and occurs irregularly throughout the daytime hours.), Advanced Sleep Phase Syndrome (characterized by intractable sleepiness during the early evening hours with awakening typically between 2 and 4 am), Non-24-hour Sleep/Wake Syndrome (characterized by intermittent insomnia that recurs with a regular periodicity over several days), and Time Zone Change Syndrome (jet lag). In addition, the modulators can be administered to, e.g., persons who live in a climate or climates which possess abnormal amounts of light or darkness; those suffering from winter depression, or other forms of depression; the aged; Alzheimer's disease patients, or those suffering from other forms of dementia; or patients who require dosages of medication at appropriate times in the circadian cycles. In some embodiments, the Rora modulators administered to a subject in need thereof are not melatonin or melatonin derivatives such as those described in EP585206.

In some embodiments, the subject mammal is a human. Although the present invention is applicable to both old and young people, it may find greater application in elderly people. Further, although the invention can enhance the sleep of healthy people, it can be especially beneficial for enhancing the sleep quality of people suffering from sleep disorders or sleep disturbances. In some embodiments, animals, including agriculturally important animals such as bovines or pigs, can be treated with Rora modulators.

III. Identification of Rora Circadian Rhythm Modulators

A. Assays for Rora Activity

The activity of Rora polypeptides can be assessed using a variety of in vitro and in vivo assays to determine functional, chemical, and physical effects, e.g., measuring potential modulator binding and the like. Cell based assays can be used to identify Rora modulators by detecting a change in a Rora-mediated activity in a cell contacted with a potential modulator. For example, as described herein, Bmal1 is positively regulated by Rora. Thus, Bmal1 expression (e.g., via monitoring with a reporter gene operably linked to the Bmal1 promoter) in a cell can be detected in the presence or absence of either Rora (e.g., transfected into the same cell) or presence or absence of the modulator. A change in Bmal1 expression in the presence of both the modulator and Rora, when the change is not observed in the absence of at least one of them, indicates that the modulator is mediated by Rora.

Preliminary screens to identify potential modulators of circadian rhythm can be conducted by screening for agents capable of binding to Rora. Binding assays usually involve contacting Rora with one or more test agents and allowing sufficient time for the protein and test agents to form a binding complex. Any binding complexes formed can be detected using any of a number of established analytical techniques. Protein binding assays include, but are not limited to, methods that measure co-precipitation or co-migration on non-denaturing SDS-polyacrylamide gels, and co-migration on Western blots (see, e.g., Bennet, J. P. and Yamamura, H. I. (1985) “Neurotransmitter, Hormone or Drug Receptor Binding Methods,” in Neurotransmitter Receptor Binding (Yamamura, H. I., et al., eds.), pp. 61-89. Other binding assays involve the use of mass spectrometry or NMR techniques to identify molecules bound to a polypeptide of the invention or displacement of labeled substrates. The polypeptides of the invention utilized in such assays can be naturally expressed, cloned or synthesized. In addition, mammalian or yeast two-hybrid approaches (see, e.g., Bartel, P. L. et. al. Methods Enzymol, 254:241 (1995)) can be used to identify polypeptides or other molecules that interact or bind when expressed together in a host cell.

Methods of identifying Rora modulators for use for treatment of other disorders are described in, e.g., U.S. Pat. No. 5,958,683. These methods can be adapted for identifying circadian rhythm modulators. For example, the expression from a Rora response element can be operably linked to a reporter gene can be monitored in a cell expressing Rora and monitored for increased reporter activity following exposure to a modulator. An exemplary Rora response element is GTAGGTCATGACCTAC (SEQ ID NO:1).

The Rora of the assay can be any Rora polypeptide or a conservatively modified variant thereof. Alternatively, the Rora polypeptides will be derived from a eukaryote and be substantially identical to any known Rora protein. Generally, the amino acid sequence identity will be at least 70%, optionally at least 85%, optionally at least 90-95% to a human Rora. Optionally, the polypeptide of the assays will comprise a domain of Rora. Either Rora or a domain thereof can be covalently linked to a heterologous protein to create a chimeric protein used in the assays described herein. Rora polypeptides are typically expressed via recombinant DNA technology in a cell.

Modulators of Rora activity are tested using either recombinant or naturally occurring Rora polypeptides. The protein can be isolated, expressed in a cell, expressed in a membrane derived from a cell, expressed in tissue or in an animal, either recombinant or naturally occurring. Modulation is tested using one of the in vitro or in vivo assays described herein or as known to those in the art.

Samples or assays that are treated with a potential Rora modulator inhibitor or activator are compared to control samples without the test compound, to examine the extent of modulation. Control samples (untreated with activators or inhibitors) are assigned a relative Rora activity value of 100. Inhibition of Rora is achieved when the Rora activity value relative to the control is about 90%, optionally 50%, optionally 25-0%. Activation of Rora is achieved when the Rora activity value relative to the control is 110%, optionally 150%, 200-500%, or 1000-2000%.

To further validate identified compounds, lead candidates can be screened for an effect on circadian rhythm in animals, e.g., using the locomotor assays described herein.

B. Modulators

The agents tested as Rora modulators can be any small chemical compound, or a biological entity, such as a protein, sugar, nucleic acid or lipid. Alternatively, test compounds will be small organic molecules (e.g., less than 1000-1500 daltons) or peptides. Essentially any chemical compound can be used as a potential modulator or ligand in the assays of the invention, although most often compounds that can be dissolved in aqueous or organic (especially DMSO-based) solutions are used. The assays are designed to screen large chemical libraries by automating the assay steps and providing compounds from any convenient source. Assays are typically run in parallel (e.g., in microtiter formats on microtiter plates in robotic assays). Modulators can also include agents designed to alter the level of Rora mRNA (e.g. antisense molecules, ribozymes, DNAzymes, small inhibitory RNAs and the like). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

In some embodiments, high throughput screening methods involve providing a combinatorial chemical or peptide library containing a large number of potential therapeutic compounds (potential modulator compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14 (3):3.09-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, Jan 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

Exemplary Rora modulators include the Rora activators described in, e.g., U.S. Pat. No. 5,958,683. Exemplary Rora modulators are displayed in formulas (1) to (17): 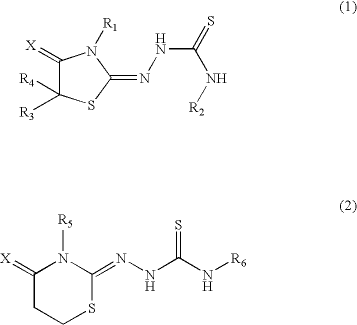
wherein
R₁ and R₅ are C₃-C₅alkyl, C₃-C₅alk-2-en-1-yl or C₃-C₅alk-2-yn-1-yl; e.g., allyl, methallyl and propinyl;
R₂ and R₆ are hydrogen, C₁-C₅alkyl, C₃-C₅alk-en-1-yl, C₃-C₅alk-2-yn-1-yl; aryl, aryl lower alkyl, saturated or unsaturated heterocyclyl lower alkyl or lower alkoxy carbonyl lower alkyl;
R₃ and R₄ are each selected independently from hydrogen and lower alkyl or together form lower alkylidene; and
X is oxo or sulfo.

Another exemplary compound is displayed in Formula 3: 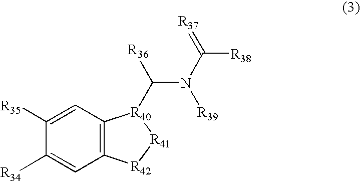
wherein R₃₄ and R₃₅ are independent of the other hydrogen, methoxy, or fluoro; R₃₆ is hydrogen or methoxycarbonyl, R₃₇ is oxo or sulfo; R₃₈ is hydrogen, C₁-C₆alkyl, cyclopropyl, cycloputyl, cyclopentyl, cyclohexyl, C₁-C₆alkyl substituted by Br, Cl, F or I, phenyl, C₁-C₃alkyl-benzene, substituted or unsubstituted by halogen, indolyl, morpholino, methylmorpholino, amino, amino substituted with C₁-C₄alkyl, or 1-(2′,3′,4′-trimethoxybenzyl)piperazine-methyl, 2-pyrrolidinone; R₃₉ is hydrogen, methyl or fluoro; R₄₀ is a carbon or nitrogen atom; R₄₁ is a carbon or nitrogen atom or a carbonyl group; and R₄₂ is a carbon, nitrogen or sulfor atom or a vinylene group. The bond between R₄₀ and R₄₁ may be a single or double bond, with the proviso that it is a single bond if R₄₁ is a carbonyl group or R₄₀ is a nitrogen atom.

Methods for the synthesis of these compounds and are given, for example in EP-447285, EP-A-494047, EP-506539, EP-A-508955, EP-527687, EP-530087, EP-A-548017, EP-A-548018, EP-562956, EP-578620, EP-A-585206, EP-591057, U.S. Pat. No. 5,283,343, U.S. Pat. No. 5,206,377, Depreux et al., J. Med Chem. 37:3231-3239 (1994), Garrat & Vonhoff, Bioorganic & Medicinal Lett. 4:1559-1565 (1994) and Copinga et al., J. Med. Chem. 36:2819-2898 (1993).

Further examples for suitable compounds are: 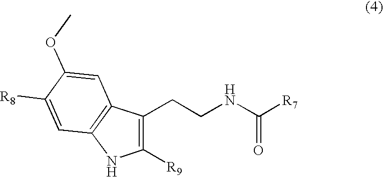
wherein
R₈=hydrogen; R₉=bromo; and R₇=methyl; or
R₈=hydrogen; R₉=iodo; and R₇=methyl; or
R₈=chloro; R₉=hydrogen; and R₇=methyl; or
R₈=hydrogen; R₉=methyl; and R₇=chloropropyl; or 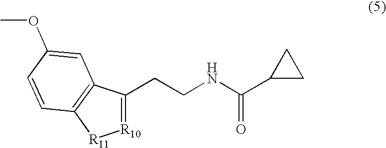
   wherein
R₁₀═CH; and R₁₁=sulfo or oxo; or
R₁₀=oxo or NH; and R₁₁═NH; or 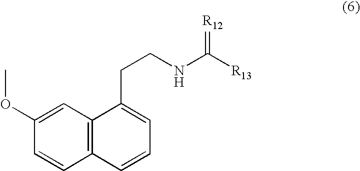
   wherein
R₁₂=oxo or sulfo; and R₁₃═NHCH₂CH₂CH₃; or
R₁₂=oxo; and R₁₃=methyl; or 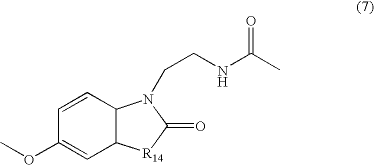
   wherein
R₁₄ is oxo or sulfo; or 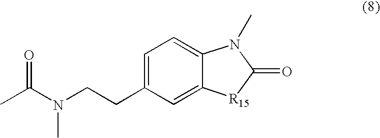
   wherein R₁₅ is oxo or sulfo; 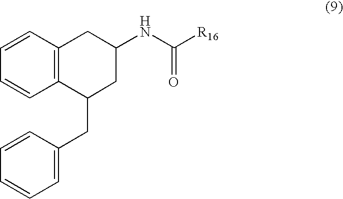
   wherein R₁₆ is methyl, ethyl or chlomethyl; or 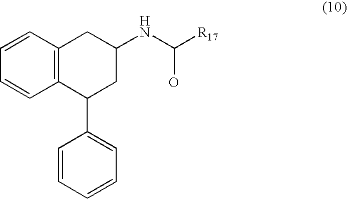
   wherein R₁₇ is methyl, ethyl or chlormethyl; or 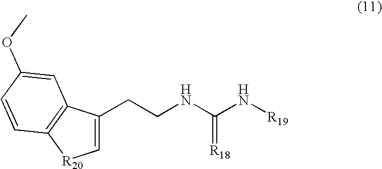
   wherein R₂₀ is NH, CH═CH, oxo or sulfo; R₁₈ is oxo or sulfo; R₁₉ is hydrogen, methyl, ethyl or propyl; or 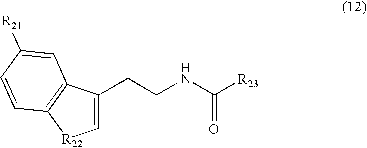
   wherein R₂₁ is methoxy or hydrogen; R₂₂ is NH, CH═CH, sulfo, or oxo; and R₂₃ is methyl, cyclopropyl or cyclobutyl; or 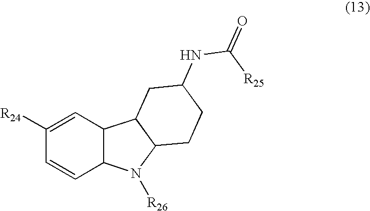
   wherein R₂₄ is hydrogen or methoxy; R₂₅ is methyl, ethyl, propyl, CF₃, CH₂Br, CHBrCH₂CH₃, cyclopropyl, or cyclobutyl; or 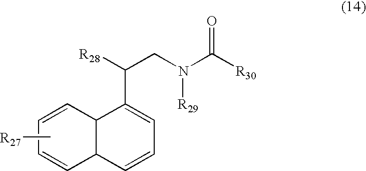
   wherein R₂₇ is methoxy; R₂₈, is hydrogen or COOCH₃; R₂₉ is hydrogen, methyl or fluoro; and R₃₀ is hydrogen, methly, ethyl, butyl, propyl, pentyl, hexyl, isopropyl, CH═CHCH₃, cyclohexyl, CH₂Br, CH₂I, CF₃, C₃H₆Cl, phenyl, 3,5-dichlorobenzene, 2-indolyl, toluene, CH(C₅H₅)₂, (CH₂)₂ C₆H₅, (CH₂)₃C₆H₅, methyl-morpholino, 1-(2′,3′,4′-trimethoxybenzyl)piperazine-methyl, 2-pyrrolidinone, SO₂CH₃; or 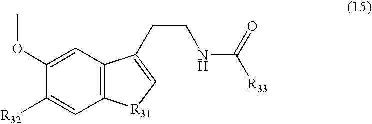
   wherein R₃₁ is NH, oxo, or sulfo; R₃₂ is hydrogen or fluoro; and R₃₃ is propyl, butyl, CH₂I, CF₃ or methyl; or 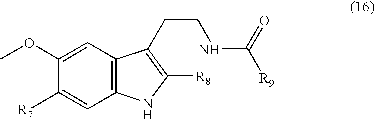
   wherein
R₇=hydrogen or C₁-C₃alkyl
R₈═C₁-C₆alkyl, aryl, hydroxy aryl or halogen; and
R₉═C₁-C₅alkyl or halogen. 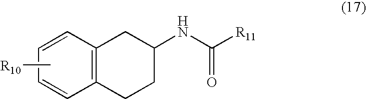
   wherein
R₁₀-hydrogen or methoxy; and
R₁₁═C₁-C₃alkyl, aryl, arylalkyl or C₁-C₃alkyl substituted with halogen. In some embodiments, the Rora modulator is described in Formula 18: 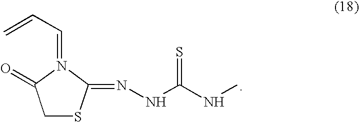
   IV. Administration and Pharmaceutical Compositions

Circadian rhythm modulators of the invention can be administered directly to the mammalian subject. Administration is by any of the routes normally used for introducing pharmaceuticals.

The pharmaceutical compositions of the invention may comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington 's Pharmaceutical Sciences, 17^{th} ed. 1985)).

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In the practice of this invention, compositions can be administered, for example, orally, nasally, topically, intravenously, intraperitoneally, intrathecally or into the eye (e.g., by eye drop or injection). The formulations of compounds can be presented in unit-dose or multi-dose sealed containers, such as ampoules and vials. Solutions and suspensions can be prepared from sterile powders, granules, and tablets of the kind previously described. The modulators can also be administered as part of a prepared food or drug.

The dose administered to a patient, in the context of the present invention should be sufficient to induce a beneficial response in the subject over time, i.e., to modulate the circadian rhythm of the subject. The optimal dose level for any patient will depend on a variety of factors including the efficacy of the specific modulator employed, the age, body weight, physical activity, and diet of the patient, and on a possible combination with other drug. The size of the dose also will be determined by the existence, nature, and extent of any adverse side-effects that accompany the administration of a particular compound or vector in a particular subject.

In determining the effective amount of the modulator to be administered a physician may evaluate circulating plasma levels of the modulator, modulator toxicity, and the production of anti-modulator antibodies. In general, the dose equivalent of a modulator is from about 1 ng/kg to 10 mg/kg for a typical subject.

For administration, modulators of the present invention can be administered at a rate determined by the LD-50 of the modulator, and the side-effects of the modulator at various concentrations, as applied to the mass and overall health of the subject. Administration can be accomplished via single or divided doses.

The modulators of the invention may be used alone or in conjunction with other agents that are known to be beneficial in altering circadian rhythms or in the enhancement of sleep efficiency. The circadian modulators of the invention and an other agent may be coadministered, either in concomitant therapy or in a fixed combination, or they may be administered at separate times. For example, the circadian modulators of the invention may be administered in conjunction with other compounds which are known in the art to be useful for suppressing or stimulating melatonin production including melatonergic agents, noradrenergic and serotonergic re-uptake blockers, alpha-1-noradrenergic agonists, monamine oxidase inhibitors, neuropeptide Y agonists or antagonists; neurokinin-1 agonists; substance P; beta-adrenergic blockers and benzodiazepines, such as atenolol; or with other compounds which are known in the art to be useful for stimulating melatonin production including tricyclic antidepressants and alpha-2-adrenergic antagonists; or with melatonin precursors such as tryptophan, 5-hydroxytryptophan, serotonin and N-acetylserotonin; as well as melatonin analogs, melatonin agonists and melatonin antagonists, or melatonin itself. In addition, the circadian modulators of the invention may be administered in conjunction with other compounds which are known in the art to be useful for enhancing sleep quality and preventing and treating sleep disorders and sleep disturbances, including e.g., sedatives, hypnotics, anxiolytics, antipsychotics, antianxiety agents, minor tranquilizers, melatonergic agents, benzodiazepines, barbituates, 5HT-2 antagonists, and the like, such as: adinazolam, allobarbital, alonimid, alprazolam, amitriptyline, amobarbital, amoxapine, bentazepam, benzoctamine, brotizolam, bupropion, busprione, butabarbital, butalbital, capuride, carbocloral, chloral betaine, chloral hydrate, chlordiazepoxide, clomipramine, cloperidone, clorazepate, clorethate, clozapine, cyprazepam, desipramine, dexclamol, diazepam, dichloralphenazone, divalproex, diphenhydramine, doxepin, estazolam, ethchlorvynol, etomidate, fenobam, flunitrazepam, flurazepam, fluvoxamine, fluoxetine, fosazepam, glutethimide, halazepam, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, oxazepam, paraldehyde, paroxetine, pentobarbital, perlapine, perphenazine, phenelzine, phenobarbital, prazepam, promethazine, propofol, protriptyline, quazepam, reclazepam, roletamide, secobarbital, sertraline, suproclone, temazepam, thioridazine, tracazolate, tranylcypromaine, trazodone, triazolam, trepipam, tricetamide, triclofos, trifluoperazine, trimetozine, trimipramine, uldazepam, valproate, venlafaxine, zaleplon, zolazepam, zolpidem, and salts thereof, and combinations thereof, and the like.

The circadian modulators of the invention may be administered in conjunction with the use of physical methods such as with light therapy or electrical stimulation. In particular, the Rora modulators of the invention may be administered in conjunction with scheduling bright light administration, ordinary-intensity light exposure, or exposure to dim-light or darkness (or sleep).

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### EXAMPLE

Here we describe a genomics-based approach to identify novel regulators of the core oscillator. By utilizing temporal gene expression profiling of multiple tissues, cell-based functional assays and behavioral analysis, we identified the orphan nuclear receptor, Rora, as a key transcriptional activator of the circadian clock. Rora is required for normal activity rhythms through activation of the Bmal1 promoter in the SCN. Our results suggest that opposing activities of Rora and Rev-erb α, which represses Bmal1 expression, are important factors in the maintenance of circadian clock function.

Genome-wide gene expression analyses were performed using high-density DNA microarrays to identify rhythmically-expressed genes in the mouse SCN, liver, and heart, as well as kidney and aorta. Of the hundreds of cycling genes, approximately fifty displayed cyclical expression across multiple tissues (“cross-tissue cycling genes”). See Table 1.

TABLE 1
List of gene with circadian expression patterns
across multiple tissues.

| | | MMC-β values | | | |
|---|---|---|---|---|---|
| Probeset | Gene Name | Aorta | Liver | Kidney | SCN |
| 100708_at | H3f3b | 0.082927 | 0.024185 | 0.00073957 | 0.057395 |
| 93694_at | mPer2 | 0.014939 | 0.02964 | 0.0040476 | 0.095152 |
| 98111_at | Hsp105 | 0.020929 | 0.091718 | 0.0011352 | 0.10098 |
| 99076_at | Rev-erb b | 0.02623 | 0.024133 | 0.0090946 | 0.060024 |
| 100081_at | Stip 1 | 0.014902 | 0.23131 | 0.0023279 | 0.048579 |
| 92809_r_at | Fkbp4 | 0.040505 | 0.071484 | 0.0019714 | 0.072658 |
| 102955_at | Nfil3 | 0.049951 | 0.03662 | 0.0015833 | 0.20733 |
| 92821_at | Usp2/Ubp-t | 0.031205 | 0.029948 | 0.001601 | 0.49579 |
| 93496_at | RIKEN cDNA 1110059L23 gene | 0.039531 | 0.026581 | 0.0046806 | 0.17102 |
| 98507_at | Rev-erb a | 0.012533 | 0.031187 | 0.040145 | 0.065875 |
| 93772_i_at | expressed sequence AI227013 | 0.063379 | 0.10135 | 0.007008 | 0.034207 |
| 95419_at | H1f0 | 0.021491 | 0.12588 | 0.0078151 | 0.079108 |
| 94420_f_at | mCry1 | 0.014094 | 0.095182 | 0.0018381 | 0.89712 |
| 95424_at | Smt3h1 | 0.38308 | 0.0179 | 0.0019439 | 0.18314 |
| 103029_at | Pdcd4 | 0.023035 | 0.072622 | 0.036161 | 0.040629 |
| 97402_at | Temt | 0.071279 | 0.10905 | 0.0020385 | 0.19641 |
| 94796_at | ESTs | 0.29193 | 0.023142 | 0.0053198 | 0.15634 |
| 95660_at | RIKEN cDNA 0610025L15 gene | 1 | 0.014247 | 0.0021597 | 0.20562 |
| 97261_at | Dnaja1 | 0.1314 | 0.036667 | 0.0055079 | 0.31973 |
| 104701_at | Stra13 | 0.67323 | 0.022253 | 0.0050383 | 0.11672 |
| 99064_at | Usp4 | 0.058841 | 0.08681 | 0.014785 | 0.19906 |
| 98543_at | Ctss | 0.17806 | 0.076285 | 0.0059723 | 0.21148 |
| 104082_at | RAB12 | 0.18365 | 0.15858 | 0.0014011 | 0.44437 |
| 100555_at | Dscr1 | 0.49812 | 0.023476 | 0.01897 | 0.083984 |
| 99951_at | RORC | 0.13325 | 0.076255 | 0.0022559 | 1 |
| 102382_at | Mop3 | 0.12724 | 0.34715 | 0.0083243 | 0.072599 |
| 95657_f_at | D13Wsu177e | 0.24408 | 0.34137 | 0.0050683 | 0.066394 |
| 95057_at | Herpud1 | 0.32286 | 0.078197 | 0.0042533 | 0.26443 |
| 99978_s_at | Mapk14 | 0.15069 | 0.055979 | 0.0087379 | 0.38601 |
| 97451_at | Mus musculus, clone MGC: 7535 | 0.16459 | 0.034242 | 0.012466 | 0.43577 |
| 94261_at | RIKEN cDNA 2900002L20 gene | 0.03643 | 0.12149 | 0.037509 | 0.18675 |
| 99532_at | Tob1 | 0.13465 | 0.11196 | 0.053737 | 0.062496 |
| 97224_at | RIKEN cDNA 5730463C12 gene | 0.4457 | 0.027123 | 0.015903 | 0.27725 |
| 97525_at | Gyk | 0.2636 | 0.1099 | 0.0050914 | 0.38853 |
| 104390_at | expressed sequence W91701 | 0.10016 | 0.36448 | 0.035583 | 0.049177 |
| 95716_at | Ywhag | 0.48439 | 0.11023 | 0.003697 | 0.4323 |
| 94917_at | Fbo8 | 1 | 0.10877 | 0.010044 | 0.086505 |
| 94343_at | ESTs | 0.080182 | 0.18162 | 0.014333 | 0.48144 |
| 99471_at | expressed sequence AI852671 | 0.46836 | 0.1263 | 0.010742 | 0.16852 |
| 99575_at | RIKEN cDNA 1810030E05 gene | 0.24303 | 0.39198 | 0.0033363 | 0.33783 |
| 95054_at | D15Wsu59e | 0.39773 | 0.077854 | 0.011796 | 0.36693 |
| 98129_at | thymosin, beta 10 | 0.057444 | 0.37796 | 0.049817 | 0.14229 |
| 97241_at | RIKEN cDNA 4930455J02 gene | 0.067343 | 0.040479 | 0.075096 | 1 |
| 96289_at | RIKEN cDNA 0610038F01 gene | 0.27884 | 0.25502 | 0.015043 | 0.19755 |
| 93793_at | expressed sequence AA408629 | 0.026624 | 0.029571 | 1 | 0.41448 |
| 95702_at | RIKEN cDNA 1300006C19 gene | 0.386 | 0.49291 | 0.0063057 | 0.28646 |
| 95405_at | Mesdc2 | 0.49264 | 0.40074 | 0.0042281 | 0.46289 |
| 97304_at | Ubp1 | 0.35852 | 0.41295 | 0.042945 | 0.077407 |
| 94499_at | Mgea5 | 0.47029 | 0.27319 | 0.023775 | 0.18699 |
| 98447_at | C/EBPa | 0.0082122 | 0.091431 | 1 | 0.76194 |
| 93315_at | Mapk14 | 0.40141 | 0.048914 | 0.084901 | 0.51215 |
| 97900_at | Apacd | 0.4071 | 0.48763 | 0.028669 | 0.45804 |
| 101515_at | Acox1 | 0.30835 | 0.49887 | 0.039531 | 0.48144 |
| 94378_at | RGS16 | 1 | 0.12929 | 1 | 0.0272 |
| 101007_at | Mknk2 | 0.34173 | 0.02035 | 1 | 1 |
| 101889_s_at | RORA | 1 | 0.82501 | 1 | 0.07752 |

We hypothesized that cross-tissue cycling genes may function as core oscillator components. With this in mind, we performed functional cell-based screens to test the roles of these candidate genes on circadian clock activity. First, DNA microarrays were used to profile gene expression patterns from four mouse tissues, which identified a subset of 55 genes that cycled across the majority of tissues, including SCN, liver, kidney and aorta. Full-length cDNAs of cross-tissue cycling genes were then transfected with transcriptional reporters into HeLa cells to identify functional roles on clock component expression. Of the cDNAs with significant activator or repressor effects on the expression of the core components Bmal1 and Per1, genetic animal models with mutations in the cross-tissue cycling genes were then tested for defects in circadian locomotor activity rhythms. Thus, genes that fulfill the criteria of this screen share the same characteristics of most known core oscillator components.

Of the 47 cross-tissue cycling genes, we obtained 30 corresponding full-length cDNA clones. In addition, 9 genes with circadian expression patterns in 3 out of 4 tissues, as well as related family members with circadian expression in at least one other tissue. In mammals, the core oscillator resides within the hypothalamic suprachiasmatic nucleus (SCN), which can maintain circadian rhythms in the absence of synchronizing light input (Reppert, S. M. & Weaver, D. R. Nature 418:935-41 (2002)). The transcriptional activators, Clock and Bmal1, heterodimerize on E-box DNA elements within the promoters of the Period and Crytpochrome genes (Gekakis, N. et al. Science 280:1564-9 (1998); Hogenesch, J. B., et al., Proc Natl Acad Sci USA 95:5474-9 (1998); Etchegaray, et al. Nature 421:177-82 (2003)). HeLa cells were transfected with individual cDNAs and one of two transcriptional reporter constructs, Per1::luc (Gekakis, N. et al. Science 280:1564-9 (1998)) or Bmal1::luc. In addition, the Per1 luciferase reporter was co-transfected with Clock and Bmal1 expression plasmids, which resulted in over 3-fold greater transcriptional activity over reporter alone (data not shown). As expected, co-transfection of two known Per1 repressors, Cry1 and Stra13/Decl, resulted in over 6-fold reduction of Clock/Bmal1-mediated activation of the Per1::luc reporter, providing proof-of-concept for our screening assay.

Surprisingly, the retinoic acid-related orphan nuclear receptor (Ror) family member, Rorc, but not Rora and Rorb, activated Per1 expression over 8-fold, despite the lack of a consensus ROR-binding DNA element in this reporter. From the cell-based screen to identify regulators of Bmal1 expression, three cDNAs activated the Bmal1::luc reporter greater than three-fold. Co-transfection of Rora and Rorc resulted in approximately 16- and 5-fold higher Bmal1 expression levels, respectively, over the empty expression vector. Co-transfection of Rorb failed to alter Bmal1 reporter expression, perhaps due to its inactivity in cell lines of non-neuronal origin (Greiner, E. F. et al. Proc Natl Acad Sci USA 93:10105-10 (1996)). In addition, a significant increase, greater than 40-fold, in luciferase activity was seen with co-transfection of the CCAAT/Enhancer-binding protein α (C/ebpα). Importantly, co-transfection of Rora, Rorc, or C/ebpα with empty pGL3-Basic or pGL3P reporters did not activate luciferase expression (data not shown).

A hallmark characteristic of core oscillator components is their requirement for rhythmic consolidation of locomotor activity. Defects in these components can be manifested as an alteration in rhythm period length or a complete loss in rhythmic activity (arrhythmicity). Thus, we monitored the wheel-running activity patterns of StraI3^{-/-} and staggerer mice, which contain a frame-shift mutation that truncates the Rora gene product (Hamilton, B. A. et al. Nature 379:736-9 (1996)). Mutant strains representing Rorc (Ueda, H. R. et al. Nature 418:534-9 (2002)) or C/ebpα (Wang, N. D. et al. Science 269:1108-12 (1995)) were not tested because of absent SCN expression or lethality, respectively. Wild-type, Stra13^{-/-} and staggerer siblings were first entrained in 12 hr light:12 hr dark (LD) conditions, and then allowed to free-run in constant darkness (DD). Stra13 null mutants displayed locomotor activity period lengths and phase-delay responses to a 15 minute white light pulse that were indistinguishable from their wild-type siblings, indicating that Stra13 function alone is not required for core oscillator function. Homozygous staggerer mutants had reduced levels of overall activity (as number of wheel rotations per day) compared to their wild-type and heterozygous siblings. This was expected, as staggerer mice display a cerebellar ataxia phenotype resulting from defective Purkinje-cell development (Hamilton, B. A. et al. Nature 379:736-9 (1996)).

However, even with reduced activity, we were able to observe light-induced suppression of activity of the staggerer mutants in LD. In free-running conditions, staggerer mice had two distinct phenotypes. A subset of mutants had detectable locomotor rhythms with a statistically significant, shortened locomotor activity period length of 23.16±0.18 hrs. In contrast, wild-type and heterozygous siblings had average period lengths of approximately 23.88±0.09 hrs. Free-running activity rhythms could not be detected in the five remaining mutants. To confirm the behavioral phenotypes of staggerer mutants, we assayed their locomotor activity rhythms by infra-red beam splitting. Through this method, the total daily activities of staggerer mice in entraining and free-running conditions were not statistically reduced compared to wild-type siblings. As with the wheel-running experiments, staggerer mutants entrained in LD by IR-beam splitting. Furthermore, while wild-type mice displayed rhythmic activity in DD, distinct locomotor rhythms could not be detected from staggerer mutants. Importantly, Bmal1^{-/-} null mutant mice also display arrhythmic locomotor activity (Bunger, M. K. et al. Cell 103:1009-17 (2000)), suggesting a genetic link between Rora and Bmal1 functions.

Ror and Rev-erb proteins are members of the orphan nuclear receptor family. Rev-erb α, its paralog Rev-erb β, and the Rors contain DNA-binding domains that directly interact with ROR elements (RORE sequence: A[A/T]NT[A/G]GGTCA; where N is any nucleotide) as monomers (Jetten, A. M., et al. Prog Nucleic Acid Res Mol Biol 69:205-47 (2001)). However, while Rors activate transcription, Rev-erbs potently repress transcription through interactions with nuclear co-repressors. Both Rev-erb α and Rev-erb β expression has been shown to cycle in the SCN, liver, heart (Preitner, N. et al. Cell 110:251-60 (2002); Ueda, H. R. et al. Nature 418:534-9 (2002); Panda, S. et al. Cell 109:307-20 (2002); Storch, K. F. et al. Nature 417:78-83 (2002)), kidney and aorta. Interestingly, circadian expression of Bmal1, which harbors a consensus RORE in its promoter, is in antiphase to Rev-erb α expression and nearly in phase with Rora expression in the SCN and Rorc expression in the liver and kidney. Moreover, Rev-erb α activity appears to be responsible for trough levels of circadian Bmal1 expression. Mice with loss-of-function deletions in the Rev-erb α gene express constitutively elevated levels of Bmal1 mRNA, which may cause shortened locomotor period length rhythms. Prior to our results, the precise trans-activators that drive Bmal1 expression were unknown.

Surprisingly, no cDNA repressed Bmal1::luc reporter activity, which may have resulted in the exclusion of repressor elements from the 530 bp promoter region. Alternatively, basal reporter activity was less than two-fold greater in untransfected cells (data not shown) and thus, our minimal criteria of at least three-fold changes in reporter activity could not be met. This was likely why Rev-erb α, which functions on the Bmal1 ROR element, was not identified in the cell-based assay. Therefore, we addressed this possibility by testing the ability of Rev-erb α to functionally antagonize Rora activity on the Bmal1 promoter. A previous study identified four Rora isoforms (Rora1-4), of which Rora4 was used in our initial cDNA screen. While Rora2 and 3 isoforms are expressed exclusively in testes, both Rora1 and Rora4 are expressed in the brain and peripheral tissues, such as the liver. However, Rora1 is the sole variant expressed in the thalamus, the region of the brain where the SCN resides. Therefore, we assessed the ability of Rev-erb α to antagonize Rora1 activity on the Bmal1 promoter in co-transfection assays. As with the Rora4 and Rorc, transfection of increasing amounts of the Rora1 expression plasmid resulted in dose-dependent activation of Bmal1::luc reporter. The additional co-transfection of increasing amounts of the Rev-erb α expression plasmid with the Rora1 cDNA resulted in the dose-dependent reduction in Bmal1 reporter activity. Rev-erb β also antagonized Rora1 activity, while both Rev-erb α and β similarly antagonized Rora4 and Rorc activities.

One mechanism by which functional antagonism between Ror and Rev-erb activities could occur is through competitive binding of the ROR element in the Bmal1 promoter. Indeed, Ror activity on the Bmal1 reporter is dependent upon the ROR element. A single mutation in the consensus RORE resulted in at least two-fold reduction in Ror activity on the Bmal 1 reporter. We further tested this hypothesis by performing electrophoretic mobility shift assays (EMSA) with a radiolabeled DNA probe containing the Bmal1 RORE incubated with in vitro transcribed/translated Rora1, Rora4, Rorc, Rev-erb α or Rev-erb β. All five orphan nuclear receptors formed specific complexes with the Bmal1 ROR probe, which were not seen with reticulocyte lysates incubated with empty vector. Importantly, these complexes were competed by excess unlabeled RORE oligonucleotide, while an oligonucleotide containing the same RORE mutation used in transfection assays did not compete with the wild-type probe for binding the specific complexes. Thus, like Rev-erb α, Rora1, Rora4, Rorc, and Rev-erb β can form specific complexes on the Bmal1 ROR element. Furthermore, we directly tested whether Rora1 can physically compete with Rev-erb α by performing EMSAs with a fixed amount of Rora1 and increasing amounts (0.25, 0.5, 1 and 2-fold molar excess over Rora1) of Rev-erb α. The addition of higher amounts of Rev-erb α resulted in increased formation of the Rev-erb α complex on the Bmal1 probe, along with the progressive reduction in Rora1 binding to the ROR element. Together, the functional and EMSA results suggest that Rora1 and Rev-erb α can compete for binding to the ROR element to regulate Bmal1 expression.

Our molecular and behavioral findings suggest that Rora functions in the positive limb of circadian Bmal1 expression in the SCN. Circadian Rora expression in the SCN, which peaks near the time of maximal Bmal1 expression levels, may drive Bmal1 expression, while peak levels of Rev-erb α at 8-12 hours earlier may maintain the nadir of Bmal1 levels. To determine the requirement for Rora on Bmal1 levels in the SCN, we performed in situ hybridization on coronal sections of mouse brains from wild-type and staggerer mutant mice at CT6 and CT18, the times of peak and trough Bmal1 expression. Although Bmal1 expression still appeared to cycle, the levels of Bmal1 transcript in the SCN of staggerer mutants were significantly reduced compared to wild-type mice at both time-points. In addition, we tested whether circadian expression was altered in the liver of staggerer mutants. Total mRNA harvested from wild-type or staggerer liver at CT6 and CT18 was profiled with Affymetrix micro-arrays. While Cryl and Per2 did not significantly change, peak expression of Per1 (Student's t-test, P<0.001) at CT18 was reduced. Interestingly, expression of D-site binding protein (Dbp), a known Clock/Bmal1 target, was significantly higher at CT6 (Student's t-test, P<0.016), the time of trough expression in wild-type liver, than at CT18. No significant change in Bmal1 expression was observed, however this may be maintained by unaltered Rorc levels in staggerer liver (data not shown). These variations in clock-controlled gene expression patterns in the liver possibly reflect differential sensitivities to SCN function or requirements for non-circadian Rora expression in the liver.

Upon examination of the Rora expression pattern in the SCN, we found that its phase closely resembled that of two known Clock/Bmal1 targets, Per1 and Per2. Therefore, we profiled Rora mRNA levels in the hypothalamus or SCN of wild-type, Bmal1^{-/-} and Clock mice near the peak time of Rora expression. Reduced levels of Rora expression were found in the Bmal1^{-/-} hypothalamus (Student's t-test, P<0.012) and the Clock SCN (Student's t-test, P<0.13). Thus, Bmal1 and likely Clock appear to be necessary for normal Rora expression.

Our results identify an inter-play between orphan nuclear receptors with opposing transcriptional activities that maintain the appropriate Bmal1 expression levels in the SCN at specific times of the day. Interestingly, mouse Rorb null mutants have long period phenotypes (Becker-Andre) and circadian Rorb expression has been observed in the SCN. However, circadian Rorb expression peaks at CT4, the time of trough Bmal1 expression. Thus, it is unclear whether Rorb directly regulates Bmal1 expression. Recently, a similar transcriptional regulatory mechanism was uncovered in the Drosophila circadian clock. In flies, dCLOCK (dClk) and the Bmal1 homologue, CYCLE (CYC), drive the circadian expression of the dCLK/CYC repressors, dPERIOD (dTIM) and dTIMELESS (dTIM). In contrast to the mammalian oscillator, dCLK expression is cyclical, while CYC expression remains constant. Moreover, circadian dCLK expression is driven by cyclical and reciprocal activities of the basic leucine zipper transcription factors, VRILLE (VR1) and PDP1. Both VRI and PDP1 can bind to a near consensus VRI/PDP 1-binding site within the dClk promoter to repress or activate transcription, respectively. As with Rev-erb α and Rora, mutations in vri and pdp1 affect rhythmic locomotor activity and dCLK expression levels. Furthermore, both circadian vri and pdp1 expression requires dCLK/CYC, thereby interconnecting the dPER/dTIM and VRI/PDP1 feedback loops. Similarly, normal Rora and Rev-erb α expression also depends upon Clock and Bmal1, however their direct requirement is unknown.

Here we describe a novel phenotype-driven “forward genomics” strategy towards the elucidation of circadian clock regulation in mammals. Reasoning that bonafide clock components i) cycle at the transcriptional level in multiple tissues, ii) can functionally regulate the activities of the Per1 or Bmal1 promoters, and iii) are required for circadian locomotor activity, we utilized RNA expression profiling, functional cell-based screening, and behavioral analysis of mutant mouse strains to identify genes meeting these criteria. While each of these analytical methods alone cannot implicate a gene as a clock component, this lines-of-evidence approach can be an effective and efficient means to prioritize genes for their potential roles in the core oscillator.

Methods

Gene profiling analysis. mRNA extraction from mouse liver, kidney and aorta, labeling and hybridization to high-density oliognucleotide arrays were performed as described elsewhere (Panda, S. et al. Cell 109, 307-20 (2002)). Gene expression profiles from these tissues are publicly available through the Internet (http://expression.gnf.org/circadian). Identification of genes with circadian expression patterns was performed by a cosine wave-fitting algorithm, COSOPT (Panda, S. et al. Cell 109, 307-20 (2002)), which assigns a multiple measures corrected minus β (MMC-β) value indicating the goodness-of-fit for a gene expression pattern to an approximate 24-hr cosine wave. MMC-β values <0.1 have been previously assigned to genes with circadian expression patterns (Panda, S. et al. Cell 109, 307-20 (2002)). Genes with MMC-β values of <0.5 across all four tissues were defined as putative cross-tissue cycling genes. In addition, 8 genes with MMC-β values <0.2 in 3 out of 4 tissues were also included to compensate for low signal-to-noise ratios in one of the four tissues. Total mRNA from individual adult wild-type and staggerer livers at CT6 and CT18 were homogenized and extracted in 1 ml of Trizol (Invitrogen), and then purified with RNeasy miniprep columns (Qiagen). 5 ug of mRNA from individual livers were labeled, hybridized on custom Affymetrix GNF1M gene chips, washed and scanned as described above. For Clock and Bmal1 mutant profiling, wild-type and mutant mice were entrained for 1 week in LD, then sacrificed at CT10 and CT8, respectively. Clock SCN and Bmal1 hypothalamus were then dissected and total mRNA harvest as above. Two replicates of 100 ng mRNA pooled equally from 4 mice were amplified with Superscript II cDNA synthesis kit (Gibco). Generated cDNA was the purified with the Qiaquick PCR product purification kit (Qiagen). Purified cDNA was then in vitro transcribed by MEGAscript kit (Ambion). Generated cRNA was purifed with Rneasy columns, and then subjected to a second round of Superscript II cDNA synthesis. Doubly-amplified cDNA was purified as above and labled cRNA was generated with the Label Transcription kit (Enzo Diagnostics, Inc.). Labeled cRNA was then hybridized to Affymetrix U74A gene chips, washed, and scanned as described.

Plasmid construction. 530 base-pairs of the Bmal1 promoter starting at 442 base-pairs upstream and ending 108 base-pairs downstream of the transcriptional start was PCR-amplified by Expand Long Template PCR system (Roche) from C57B1/6 mouse genomic DNA with primers containing flanking Xho I or Sac I restriction sites: 5′-GATCGAGCTCGGGACGACGGCGAGCTCGCAGAG-3′,5′-GATCCTCGAGCGCACCCGCACTCGGATCCCG-3′. Primer designs were based upon published Bmal1 promoter sequences (Preitner, N. et al. Cell 110:251-60 (2002); Yu, W., et al. Biochem Biophys Res Commun 290:933-41 (2002)). The PCR product was gel-purified with Mini-elute purification kit (Qiagen), digested with Xho I/Sac I enzymes and ligated into a identically-cut pGL3Basic luciferase reporter vector (Promega) to generate the Bmal1::luc reporter. cDNAs from GNF clone collection were cloned into the pCMV-Sport6 vector (Invitrogen). All reporter constructs and cDNAs were verified by sequencing. Construction of the Per1::luc reporter (Gekakis, N. et al. Science 280:1564-9 (1998)) and Clock expression plasmid (McNamara, P. et al. Cell 105:877-89 (2001)) are described elsewhere.

Cell culture and cell-based transcription assays. HeLa cells (American Type Culture Collection) were maintained in Dulbecco's modified Eagle's minimal essential medium (DMEM; Gibco), 10% fetal bovine serum (FBS; Gibco), 0.1 mM non-essential amino acids (NEAA; Gibco), and Penicillin/Streptomycin/Glutamine (PSG; Gibco) at 37° C. with 5% CO₂. The day before transfection, Hela cells at 80% confluence were plated onto sterile 96-well Costar polystyrene flat bottom plates (Corning Inc.) at 2×10⁴ cells/well. The following day, the following plasmids were aliquoted as appropriate into eppendorf tubes: 25 ng luciferase reporter, 25 ng pCMV-Beta (Clontech), 50 ng pCMV-Clock, 50 ng pCMV-Bmal1, and 25, 50, or 100 ng cDNA from the GNF clone collection. The pCMV-Sport6 plasmid was used as a filler to bring the total DNA concentration to 250 ng/well. For transfections with Bmal1::luc, 100 ng of pCMV-Sport6 plasmid was used in place of Clock/Bmal1. In dose-dependent assays, 25, 50, or 100 ng of plasmid was transfected. For competition and all other assays, expression plasmids were used at 100 ng/well. The plasmids were brought to a total volume of 30 μl with DMEM, mixed with 20 ul of 1:20 Polyfect (Invitrogen):DMEM and then incubated at room temperature for 10 minutes. After incubation, 100 ul of DMEM/FBS/NEAA/PSG was added to the DNA, transferred onto PBS-washed HeLa cells in the 96-well plate, and then incubated at 37° C./5% CO₂. Each DNA condition was conducted in triplicate for each transfection experiment. After 24 hrs, transfected cells were washed with PBS and assayed for luciferase and beta-galactosidase activities with Dual Light Kit (Tropix) according to manufacturer's specifications. Luminescence counts were measured with an Acquest machine (LJL Biosystems). Triplicate ratios of luciferase activity to beta-galactosidase activity from individual transfections were averaged and fold-activations were calculated within each experimental event. Large-scale transfection screens were performed twice, while all other assays were performed at least three times. All cDNA hits from the cell-based screens were sequence-verified.

Locomotor activity assays and analysis. All animal procedures were approved by the AALAC of GNF, San Diego, Calif. Rora^{sg/+} mice after 9 generations of backcrosses to C57B16 were bred, and the progeny were genotyped. Stra13^{-/+} mice of 129S/C57B16 mixed background were bred and the progeny were genotyped. 8-16 week old mice were individually housed in running-wheel equipped cages placed in light-tight chambers at constant temperature (22° C.). Mice were entrained to 12 hrs of white light (800 lux white fluorescent) and 12 hrs of darkness for 10-15 days, and released into constant darkness (DD). After 17-20 days in DD, mice receiving light stimulus received a single 15 min pulse of white light at 4 hours after activity onset. Mice received food and water ad libitum. Rora^{sg/sg} mice were ensured additional pre-wet food inside the cage. Experiments were monitored using Clocklab (Actimetrix) software. For IR-beam splitting assays, activity rhythms of 4 wild-type and 4 staggerer male mice were assayed by the MicroMax home cage monitoring system (Accuscan Instruments, Inc.). The number of IR-beam splits was recorded in 20-minute bins for 9 days in LD and 9 days in DD. Analyses of wheel-running and IR-beam splitting data were performed with Clocklab and Matlab 11.1.

Electrophoretic mobility shift assays (EMSA). The following complementary oligos (Gibco) were annealed to generate probes representing the ROR element (underlined, bold nucleotide denotes mutation site) of the Bmal1 promoter: Bmal1 RORE wild-type, 5′-GAAGGCAGAAAGTAGGTCAGGGACGGAG-3′ and 5′-CTCCGTCCCTGACCTACTTTCTGCCTTC-3′, Bmal1 RORE mutant, 5′-GAAGGCAGAAAGTACGTCAGGGACGGAG-3′ and 5′-CTCCGTCCCTGACGTACTTTCTGCCTTC-3′. Annealed wild-type RORE oligos were labeled with polynucleotide kinase (New England Biolabs) and γ[³²P]dATP. Labeled probes were phenol-chloroform extracted, and then purified twice with MicroSpin G-25 columns (Amersham Pharmacia). One fmole of labeled probe was incubated with in vitro transcribed/translated (TNT) reticulocyte lysate in 10 mM Hepes, pH 8.0, 1 mM EDTA, 50 mM KCl, 5 mM MgCl, 5% glycerol, 0.5 mM dithiothreitol, 2.5 ug poly(dIdC), 1× Complete protease inhibitor (Gibco) for 10 minutes at room temperature. In vitro transcription/translation of pCMV-Sport6, pCMV-Rora, pCMV-Rorc and pCMV-Rev-erb β plasmids were performed with TNT SP6 Quick Coupled Transcription/Translation System (Promega) according to manufacturer's specifications. After incubation, protein-DNA complexes were separated by non-denaturing 5%-acrylimide gel electrophoresis at 4° C. and visualized by phosphorimaging. One picomole of unlabeled wild-type or mutant Bmal1 RORE oligos were incubated with radio-labeled oligonucleotide and reticulocyte lysate in competitive EMSAs. TNT protein quantification was performed by translating in parallel with ³⁵S-methionine, separating labeled protein by SDS polyacrylimide gel electrophoresis (SDS-PAGE), and then equalizing amounts of translated protein by phosphorimaging.

Hybridization histochemistry. In situ hybridization was performed using an ³⁵S-labeled antisense CRNA probe generated from nucleotides 864 to 1362 of mouse Bmallb (Shearman et al.). Brains from adult wild-type and staggerer mice entrained for 7 days were removed at CT6 and CT18 and fixed in formalin for 10 days at 4° C. Fixed brains were then embedded and frozen in OCT (Sakura Finetech). Serial coronal brain sections of 12 μm in thickness were placed and dried on glass slides. Sections were then digested with 0.1-10 μg/ml proteinase K for 30 min at 37° C. Probes were labeled to specific activities of 1-3×10⁹ dpm/μg, and applied to the slide at concentrations of about 107 cpm/ml, overnight at 56° C. in a solution containing 50% formamide, 0.3 M NaCl, 10 mM Tris, 1 mM EDTA, 0.05% tRNA, 10 mM dithiothreitol, 1× Denhardt's solution and 10% dextran sulfate, after which they were treated with 20 μg/ml of ribonuclease A for 30 min at 37° C. and washed in 15 mM NaCl/1.5 mM sodium citrate, at 60° C. Slides were then dehydrated and exposed to x-ray films (B-Max; Kodak) for 72 hr. They were coated with Kodak NTB-2 liquid emulsion and exposed at 4° C. for 45 days. Slides were developed with Kodak D-19 and fixed with Kodak rapid fixer.

The above example is provided to illustrate the invention but not to limit its scope. Other variants of the inventions will be readily apparent to one of ordinary skill in the art and are encompassed by the appended claims. All publications, databases, Genbank sequences, patents, and patent applications cited herein are hereby incorporated by reference.

1. A method for identifying a therapeutic agent for modulating circadian rhythm in an animal, the method comprising:
identifying an agent that modulates Rora activity or expression;
testing the identified agent for an effect on the regulation of circadian rhythm in the animal; and
selecting an agent that modulates the regulation of circadian rhythm in the animal.

2. The method of claim 1, wherein Rora is selected from the group consisting of human Rora1, human Rora2, human Rora3, and human Rora4.

3. The method of claim 1, wherein the agent increases Rora expression.

4. The method of claim 1, wherein the agent increases Rora activity.

5. The method of claim 1, wherein the agent decreases Rora expression.

6. The method of claim 1, wherein the agent decreases Rora activity.

7. The method of claim 1, wherein Rora activity is measured by determining the expression from Bmal1 promoter.

8. The method of claim 7, wherein the Bmal1 promoter is operably linked to a reporter polynucleotide.

9. The method of claim 1, wherein the animal is a mouse.

10. A method of modulating circadian rhythm in a mammal in need thereof, the method comprising administering to the mammal an effective amount of a Rora modulator.

11. The method of claim 10, wherein the modulator is by a method comprising the steps of
identifying an agent that modulates Rora activity or expression;
testing the identified agent for an effect on the regulation of circadian rhythm in the animal; and
selecting an agent that modulates the regulation of circadian rhythm in the animal, thereby identifying a modulator of circadian rhythm.

12. The method of claim 10, wherein timing of administration of the selected agent is pre-determined to coincide with an appropriate phase of an existing circadian rhythm to produce a selected modulation of the circadian rhythm.

13. The method of claim 10, wherein the selected agent is used to treat or prevent a sleep disorder.

14. The method of claim 10, wherein the mammal has a condition selected from the group selected from insomnia, Seasonal Affective Disorder, Shift Work dysrhythmia, delayed-sleep phase syndrome, and jet-lag.

15. The method of claim 10, wherein the mammal is a human.

16. The method of claim 10, wherein the selected agent is administered in conjunction with melatonin or a compound that suppresses or stimulates melatonin production.

17. The method of claim 10, wherein the selected agent is administered in conjunction with light therapy.
